(19) United States
(12) Patent Application Publication
(10) Pub. No.: US 2014/0205687 A1
(43) Pub. Date: Jul. 24, 2014

(54) TRIBULUS TERRESTRIS, AVENA SATIVA AND PANAX GINSENG EXTRACT COMBINATION

(71) Applicants: Mustafa TEKIN, Kocasinan Kayseri (TR); Mustafa ERASLAN, Ankara (TR)

(72) Inventors: Mustafa Eraslan, Ankara (TR); Mustafa Tekin, Kocasinan Kayseri (TR)

(21) Appl. No.: 14/240,738

(22) PCT Filed: Mar. 5, 2012
(86) PCT No.: PCT/TR2012/000044

§ 371 Date: Feb. 24, 2014

(30) Foreign Application Priority Data
Aug. 23, 2011 (TR) 2011/08485

(51) Int. Cl. A61K 36/185; A61K 36/258; A61K 36/899

(52) U.S. Cl. CPC A61K 36/185; A61K 36/899; A61K 36/258
USPC 424/728

(57) ABSTRACT

The present invention, is related to a herbal pharmaceutical product, obtained from the defined parts of the tribulus terrestris, avena sativa and panax ginseng plants, in order to be used as a supplement and support to increase muscle strength, body stamina and physical performance and in order to treat cardiovascular diseases and the present invention is also related to the formulation of said product.

The present invention, is related to a herbal pharmaceutical product, obtained from the defined parts of the tribulus terrestris, avena sativa and panax ginseng plants, in order to be used as a supplement and support to increase muscle strength, body stamina and physical performance and in order to treat cardiovascular diseases and the present invention is also related to the formulation of said product.

Tribulus terrestris (Caltrop) that is included in the developed combination, is a plant that is generally known as weeds, in gardens, which is an annual, flowery plant. This plant grows naturally in many places in the world. It can be found in abundance in India and Africa. It is positioned laterally on the soil. The leaves are compound leaves, with 10-16 pinnules, the flowers on the plant are small and light yellow colored. The fruit has pointed tips like horns which are 10 mm in diameter. Its roots contain alkaloid, resin, nitrogen and fatty oils; its fruits contain 5% oil, peroxidase, and diastase, a small quantity of glycoside resin, proteins and inorganic substances. The protein percentage is approximately 10-11%; and it contains fourteen amino acids, six of which are basic acids. Its stem and body is rich in terms of starch, fructose and sucrose. The carboline saponins it contains (harmine, harmaline, harman, tetrahydroharmine) which are steroidal saponins (mostly furastanol) can be extricated from the plant by using different methods and their rates can be standardized in plant extracts. One of the amino acids inside the plant which is arginine is an agent necessary for the production of sperms. The amino acids, saponins, and other beneficial materials inside the plant stand out with their strengthening and invigorating characteristics and features that increase physical performance and they also act like an aphrodisiac. People who perform intense physical activities, exercises and sports also prefer to use Tribulus in order to increase their strength, their stamina and energies. The root and fruits of Tribulus terrestris are being used as a moisturizing aphrodisiac, diuretic, appetizer, digestive, anthelmintic, expectorant, painkiller, anti-inflammatory, alterant, laxative, cardiotonic, antihaemorrhagic, lithotriptic, and tonic. Its leaves are used as astringent, diurent, aphrodisiac, depurative, anthelmenthic and tonic. Its seeds are good for firming, strengthening, treating nose bleeding, other bleeding types and ulcerative stomatitis.

The other product within the combination which is Avena sativa (wild oat) contains within its structure, carbohydrates, Proteins, avenasterols, Steroid saponins, Vitamins; B1, B2, B3, B6, E, K, Provitamins A (β-carotene) and panthothenic acids, and it contains an amount of 2% SO2 (Silisiumdioxide), iron, zinc, aluminium, manganese, cobalt, boron and iodine as minerals. The fatty oils are approximately 7, 4% and 1, 3% of this is fatty oils and 5, 7% of this is unsaturated fatty acids.

Oats are herbaceous, cereals which contain large amounts of starch. It is rich in vitamins especially B grouped vitamins, and rich in terms of minerals, calcium, magnesium, potassium, phosphorus and iron minerals. Its fruits are used to decrease cholesterol levels, they prevent depression, strengthen nerves, they are nutritive, they are good in healing wounds and can be used to prevent irritation. Oat bran and green oat prevents depression, is used as an anxiolytic and diaphoretic.

Oats lower the lipids, cholesterol, triglycerides and glucose in the blood. It strengthens and soothes nerves. Tincture prevents smoking and alcohol addiction. It is used commonly by people for treating and overcoming exhaustion, bad temper, physical incapacity, shivering, vertigo, rheumatism, gout, diarrhea, stomach flu, gastrointestinal disorders.

The other product within the combination which is Korean Ginseng (Panax Ginseng), is one of the most commonly used herbs in Far East countries. The herb also is famous and reputed to be a mystical plant according to far eastern people. Ginseng, is a plant that increases physical activities and stamina and it also increases mental (cognitive) and physical resistance. The beneficial agents inside the plant, will help increase the sperm production and also it will help overcome decrease of performance related to excessive stress and fatigue in men. The Korean Ginseng (Panax Ginseng) is the most common herb used for more than 2000 years in far eastern countries. Nowadays the Koran ginseng is not only used in far eastern countries, but also it is used commonly used all around the world. Panax ginseng is significantly different from other ginseng types as its production takes 6 years. Other ginsengs contain 8-9 different beneficial agents, whereas Panax ginseng (Korean ginseng) contains 22 different beneficial agents (ginsenosides, saponins, glycans (panaxans), polysaccharide fraction DPG-3-2, peptides, maltol, B vitamins, flavonoids and volatile oil) and antioxidants. According to a Chinese text that belongs to the first century, ginseng is defined as a healing herb that gives strength, which strengthens the mind, that increases comprehension and wisdom and it also states that its regular usage will increase the quality of life and will ensure longevity.

In the Korean patent application numbered KR 20100128016 (A) a ginseng panax extract preparation method is described. The Chinese patent application numbered CN 101343330 (A) is related to a panax polysaccharide preparation method. In the Japanese patent application numbered JP 2009263327 (A) a medicine that contains ginseng panax to treat gene deficiency is described. In the Korean patent application numbered KR 20090124268 (A) the use of a combination which comprises ginseng panax extract to treat diabetes is described.

In the German patent application numbered DE 3931147 (A1) the usage of an extract that contains Avena sativa and Fructus vitis viniverae as a sedative and in treating the decrease of libido is mentioned. In the American patent application numbered U.S. Pat. No. 5,151,498 (A) a formulation developed that contains avena sativa, to treat viral and bacterial infections, and infections due to genetic or temporary immunodeficiency is described.

In the Korean patent numbered KR 100848213 (B1) inhibition from adverse effects for the human body and to treat diseases related to the releasing of a growth hormone by using tribulus terrestris extract is provided.

The effects of each of the Tribulus terrestis, Avena sativa and Ginseng panax within the combination, are known for years to increase physical capacity, strengthening muscles, increasing sperm count, and increasing stamina.

The beneficial effects of Tribulus terrestis, Avena sativa ve Ginseng panax are increased by this developed combination. This invention ensures that bioavailability is increased by using the herbal extracts together in combination instead of using each of the herbal extracts on their own and achieving fewer benefits.

The usage of Tribulus terrestis, Avena sativa and Panax ginseng together, for their mentioned effects is quite low. The products obtained from these plants, when taken one by one, do not show their effects to a sufficient degree.

### Preparation Method:

In this composition developed for liquid and solid dosage types that are taken orally the fruit extract of Tribulus terrestris, the surface extract of avena sativa (picked when it is a green color), and the root extract of panax ginseng is taken in different amounts and magnesium stearate is added and mixed until a homogenous mixture is obtained. Other lubricants can also be used instead of magnesium stearate. The homogenous mixture is filled into hard gelatin capsules. Soft gelatin capsules can also be produced as tablets, and all types of dosage forms such as liquid or solid.

The amount of the extracts added into the mixture may change in accordance with the saponin amounts they contain. This is why each of the extract amounts shows difference within a combination. The fruit extract that carries 60% saponins of Tribulus terrestris is used between 300 mg-500 mg, extracts that carry 70% saponins can be used between 200-400 mg. The extracts that carry 10% saponins of avena sativa can be used between 150-250 mg, whereas extracts that carry 30% saponins can be used between 50-100 mg. The extract of Panax ginseng which carries 30% ginsenosides, can be used between 100-200 mg whereas the extract that carries 15% ginsenosides can be used between 200-400 mg. The extract amounts can increase or decrease with an inverse proportion according to the amounts of saponins they contain.

The saponins amounts within the combination can vary. Moreover a mixture can also be prepared by using the pure saponins of the Tribulus terrestris, Avena sativa and Panax ginseng herbs. The combination according to the present invention is obtained by mixing the pure saponins and/or extracts of the Tribulus terrestris, Avena sativa and Panax ginseng herbs.

Other plants or plant extracts that provide the same effects can also be added into the above mentioned triple combination.

The extract combination developed according to the present invention; is used to treat coronary artery blockages, carotid artery blockages, general body atherosclerosis, hyperlipidemia, hypercholesterolemia, multiplesclerosis, Alzheimer's, dementia, stroke due to embolism (seizure), Parkinson's, buergers disease, Chronic Obstructive Pulmonary Disease, type1 and type2 diabetes, to prevent and eliminate vascular complications as a prophylactic, to treat impotence, infertility (to aid in increasing sperm count, quality and sperm activity), and to aid in increasing muscle strength.

1. An herbal pharmaceutical composition for treatment of cardiovascular diseases, the composition comprising fruit extract of Tribulus terrestris, above-ground part extract of avena sativa and root extract of panax ginseng.

2. The herbal pharmaceutical composition according to claim 1, wherein the composition comprising 300-500 mg of the fruit extract of Tribulus terrestris, 150-250 mg of the above-ground part extract of avena sativa and 100-400 mg of the root extract of panax ginseng.

3. The herbal pharmaceutical composition according to claim 1, wherein the composition comprising 400 mg of the fruit extract of Tribulus terrestris 200 mg of the above-ground part extract of avena sativa and 150 mg of the root extract of panax ginseng.

4. The herbal pharmaceutical composition according to claim 3, wherein the fruit extract carries 60% saponins of Tribulus terrestris.

5. The herbal pharmaceutical composition according to claim 3, wherein the above-ground part extract carries 10% saponins of avena saliva.

6. The herbal pharmaceutical composition according to claim 3, wherein the root extract carries 30% saponins of panax ginseng.
